(19) United States
(12) Patent Application Publication
(10) Pub. No.: US 2006/0155301 A1
(43) Pub. Date: Jul. 13, 2006

(54) Methods and devices usable in eye surgery

(76) Inventor: Jörgen Holmen, Uppsala (SE)

Correspondence Address: YOUNG & THOMPSON, 745 SOUTH 23RD STREET, 2ND FLOOR, ARLINGTON, VA, 22202, US

(21) Appl. No.: 10/533,405

(22) PCT Filed: Nov. 3, 2003
(86) PCT No.: PCT/SE03/01692

§ 371 Date: Nov. 4, 2005

Related U.S. Application Data
(60) Provisional application No. 60/422,872, filed on Nov. 1, 2002.

(30) Foreign Application Priority Data
Nov. 1, 2002 (SE) 0203245-6

(51) Int. Cl. A61F 9/00

(52) U.S. Cl. 606/107

(57) ABSTRACT

A device (10) for preventing leakage of gas from a lens capsule of an eye, to which a capsulorhexis has been made, during manipulations performed inside the lens capsule. It includes a tube (12) having at least one lumen (14). The tube is provided with at least one barrier member (16; 17) such that gas that has been introduced into the lens capsule is prevented to escape therefrom. It also relates to a method of eye surgery. It includes making an incision to gain access to the interior of an eye. Making a capsulorhexis in the lens capsule. Removing the lens from the lens capsule of an eye. Sealing the capsulorhexis with a sealing device, to provide gas leakage proof sealing. Expanding the lens capsule with a gas. Performing a desired operation inside the expanded lens capsule.

The present invention relates to eye surgery, and in particular to a method and devices for allowing and facilitating efficient removal or inactivation of lens epithelial cells for the purpose of preventing Capsular Opacification.

### BACKGROUND OF THE INVENTION

The crystalline lens of the human eye is located in the posterior chamber between the posterior iris surface and the vitreous body. It is a biconvex transparent tissue without nerves and blood vessels, weighing approximately 0.2 g. The lens is enveloped in a capsule, a structureless, transparent and elastic membrane bag. Approximately 80 zonular fibers, extending between the capsule and the ciliary body, suspend the lens. The inside of the lens capsule consists of lens epithelial cells and lens fibers. The lens epithelial cells form a monolayer underlying the capsule from the anterior pole to the equator of the lens. These cells continue to undergo cell mitosis throughout life in the area located between the anterior pole and the lens equator. The lens epithelial cells that underwent cell mitosis gradually move toward the lens equator and differentiate into lens fibers. These cells make up the rest of the lens. New layers of fiber cells are constantly formed on top of those previously formed. The older fiber cells become denser and during the 3rd decade of life a hard nucleus is formed in the middle of the human lens, consisting of old dehydrated fiber cells.

A cataract is defined as every form of opacity in the lens or its capsule; the lens becomes cloudy, resulting in a loss of visual ability. A cataract is a painless phenomenon, but decreases the quality of life if the lens is not surgically extracted and replaced by an artificial lens.

When the lens is surgically extracted, an incision is made in the anterior part of the eye, i.e., the cornea or the sclera. Then, a viscoelastic material is usually introduced into the anterior chamber to maintain the anterior chamber depth during surgery. An opening is made in the lens capsule by a procedure called capsulorhexis.

Following capsulorhexis, the lens is removed according to one of two principles: extracapsular cataract extraction (ECCE)—the cataractous lens is squeezed out through an opening in the anterior lens capsule and then removed through a 10-12 mm-corneal incision, or phacoemulsification—the cataractous lens is dissolved with a special instrument, phaco-probe, by high frequency sonification and rinsed out through a 3-4 mm corneal incision.

Remaining parts of the lens, i.e. lens fibers and lens epithelial cells, are then removed using an irrigation and aspiration device. After complete removal of the lens, the lens capsule is filled with a viscoelastic material and an artificial lens is implanted into it. Alternatively, a lens can be molded inside the lens capsule, as disclosed in PCT/EP99/07780. Thereby a cross-linkable polymer is injected into a lens capsule, and the lens is formed in situ. Another method for the same purpose but employing other materials is disclosed in PCT/EP01/04010.

Dyeing of the anterior lens capsule has been used to facilitate capsulorhexis in advanced/white cataract, to enhance critical steps during phacoemulsification and to perform capsulorhexis of the posterior lens capsule. Earlier studies have evaluated dyes, such as crystal violet, fluorescein, and indocyanine green, for dyeing the anterior lens capsule. Some dyes are applied by injection under the anterior surface of the capsule. Others are applied by a certain technique in which the anterior chamber is filled by gas, and the dye is applied on top of the anterior surface of the capsule. After a while, the dye is washed away by irrigation/aspiration and the anterior chamber is filled by a viscoelastic solution followed by capsulorhexis.

After cataract surgery, the most common postoperative complication is posterior capsule opacification (PCO) which has the clinical and economic significance to be considered as an important public health problem. Studies report that the incidence of PCO is ranging from 20% to 40% after approximately 4 years after surgery. Migration and proliferation of remaining lens epithelial cells is the main cause of PCO. These cells grow from the peripheral parts of the capsule onto the posterior capsule and continue toward the axial region. Impaired visual acuity is the result caused by cell migration, proliferation and aggregation, the production of extracellular matrix, fibrosis and wrinkling of the lens capsule.

In the current clinical standard, patients who develop PCO are treated symptomatically by YAG laser capsulotomy. In this procedure a YAG laser disrupts the opacified lens capsule and the visual axis is cleared. However, YAG laser capsulotomy exposes patients to the risk of complications that can lead to severe visual impairment or loss of vision, such as retinal detachment, pupillary block glaucoma and cystoid macular edema. Other complications associated with YAG laser capsulotomy include damage to implanted intraocular lenses resulting in glare and photophobia, dislocation of intraocular lenses, iritis, vitritis, corneal edema, iris damage and rupture of the anterior hyaloid.

From an economic point of view, symptomatic treatment of PCO is ranked one of the highest of the medical costs in the U.S.A. Thus, development of a procedure to prevent PCO reduces the medical costs related to YAG laser capsulotomy, including the costs for the treatment, its complications, and YAG laser equipment. Accordingly, there is a great need for PCO prophylaxis.

Mechanical and pharmaceutical methods for PCO prophylaxis by removing or destroying residual lens epithelial cells have been developed. However, none of them has been proved to be practical, effective, and safe enough for routine clinical practice.

Capsular polishing, aspiration of residual lens epithelial cells, ultrasound combined with aspiration, cryocoagulation, and osmolysis are examples of methods that have been developed and shown to remove or destroy remaining lens epithelial cells, but none of these methods have been proven to be efficient in PCO prophylaxis.

The design of the artificial intraocular lenses (IOL), such as the shape, size and materials of the IOL implanted during cataract surgery has also been shown to affect the development of PCO. It has been shown that a sharp bend in the capsule, created by a capsule tension ring or an IOL with sharp optic edges, may induce contact inhibition of lens epithelial cell migration on the capsule.

Various anti-metabolites such as doxorubicin, methotrexate, mitomycin, daunomycin/daunorubicin, 5 fluorouracil, colchicines, and taxol are effective in inhibiting lens epithelial cells proliferation in vitro. However, in vivo animal studies have shown that there are toxic side effects in the tissues of the eye when anti-metabolites are used in sufficiently high concentration to inhibit lens epithelial cells proliferation. In attempts to avoid side effects on other ocular tissues an immunotoxin specifically inhibiting proliferation of lens epithelial cells has been evaluated. The anti-lens epithelial cell monoclonal antibody binds specifically to lens epithelial cells and carries ricin or saporin that kill proliferating cells. In the experimental studies, antibodies against human antitransferrin and FGF have been used as antibodies against lens epithelial cells. However, no conclusive results have been obtained.

Another pharmacological approach is to separate lens epithelial cells from the lens capsule. Ethylenediamine tetraacetic acid (EDTA) was included in an irrigation solution and a simulated extracapsular cataract extraction was performed to separate lens epithelial cells. In other attempts, EDTA was used with a viscoelastic material (U.S. Pat. No. 5,204,331 to Nishi et al., 1993), or simply introduced into the lens capsule. When an EDTA solution was included in an irrigation solution and a simulated extracapsular cataract extraction was performed in cadaver eyes, the anterior lens epithelial cells could be separated. EDTA seems not to be more efficient than other agents evaluated in PCO prophylaxis.

Enzymes such as trypsin and DISPOSE (protease) have also been evaluated for separation of lens epithelial cells. When a 2% trypsin solution was included in an irrigation solution and a simulated extracapsular cataract extraction was performed in cadaver eyes, lens epithelial cells were stripped in places. The cell separation was partially successful. However, the zonules were damaged by the trypsin solution. The use of an active enzyme can be a problem even when an enzyme solution is introduced into the lens capsule because it can damage the zonules bound to the lens capsule.

According to U.S. Pat. No. 4,909,784 to Dubroff 1990, when a cell-killing substance is introduced into the lens capsule through a small hole, without first removing the lens, lens epithelial cells are killed. A drawback when using this method is that the efficacy of the treatment may be strongly limited, if the natural lens is not removed before administrating the cell-killing substance. The natural lens may absorb or decrease the efficacy of the substance due to the huge number of lens epithelial cells within the lens. A viscoelastic material that is introduced into the anterior chamber prevents the active agent from escaping from the lens capsule, and prevents damage to the corneal endothelium. In related patents (U.S. Pat. No. 4,909,784 to Dubroff 1990, U.S. Pat. No. 5,013,295 to Dubroff 1991), a syringe to remove the introduced substance from the lens capsule through a small hole was disclosed. However, physically and technically, it seems to be difficult to efficiently remove the substance introduced into the lens capsule before capsulorhexis without damaging the lens capsule. The remaining substance may escape from the lens capsule and damage the cells and tissues facing the anterior chamber during and after capsulorhexis.

An important problem in connection with all methods relating to cataract surgery is the difficulty of observing the interior of the lens capsule, especially behind the iris, in order to ascertain that measure taken were successful, such as the removal of residual lens epithelial cells.

U.S. Pat. No. 5,651,783 (Reynard 1995) discloses a fiber optic sleeve that permits endoscope visualization of intraocular structures either through the surgical handpiece or through an end piece attachment. However, this patent is silent in regard of evaluating the capsular inside in a turbulent flow of irrigation solution and lens materials flowing around the end of the fiber optic during the process of phacoemulsification and irrigation-aspiration, and such evaluation appears very difficult given the premises in the patent.

Gwon et al, in J. Refract. Surgery, Vol. 19, November 1993, pp 735-746 discloses that the lens capsule was expanded with air and perfluoropropane by closing a capsulotomy of a size 2.5 to 3.5 mm with a patch, attached to the capsule by Healon and overlapping the capsule by at least 1 mm. The reason was to study the effect on lens regeneration in rabbits and cats. Nothing was explained of using the technique in other aspects. Additionally, it seems difficult to use the technique of closing off the capsule with a patch for performing different procedures within the capsule, as the patch would block introduction of devices into the capsule and performance of different methods within the capsule. Furthermore, the authors describe the situation that the capsule is not completely filled by air, but a mixture of air and viscoelastic solution (Healon®).

In Green G F et al. Prevention of posterior capsular opacification by endocapsular circulation of chemical agents (WO 02/15828), priority 21 Aug. 15, 2000, there is disclosed a tip is described for sealing a lens capsule by forming a seal with the edge of a small capsulorhexis.

In Maaloof, A et al. A device for sealing the capsular bag of an eye and a method for delivering fluid or treatment substances to the lens of an eye (WO 02/43632 A1), priority 28 Nov. 2000, 19 Apr. 2001, there is disclosed a plug is described for sealing the lens capsule and a method for flushing solutions or chemicals inside the lens capsule.

These patents are silent in regard of using a gas to expand the capsule, and also about delivering low doses of agent solutions to the lens epithelial cells situated at the capsular surface by the phenomenon of surface tension. It seems hazardous to flush or fill the capsule completely with an agent solution, because it exposes the patients' eye to a high dose of the agent solution. There is also a risk that the plug or the tip get loose so that the agent solution leaks out into the anterior chamber and injures delicate tissues, such as the corneal endothelium. A drawback with using the described plug or tip is that no devices can be introduced into the capsule after the plug or the tip has been mounted at the capsule.

U.S. Pat. No. 5,061,696 to York discloses a method for destroying lens epithelial cells comprising using a double cannula for simultaneously aspirating aqueous and/or irrigating solution from the lens capsule, posterior chamber and anterior chamber, injecting an ocularly biocompatible gas to fill the anterior chamber, posterior chamber and lens capsule with said gas, and then injecting via a separate cannula, a hypotonic viscoelastic substance to fill the lens capsule and osmotically destroy the lens epithelial cells without diluting the hypotonic viscoelastic with isotonic solutions in the eye

### SUMMARY OF THE INVENTION

In one aspect of the invention there is provided a method for the sealing of the lens capsule of an eye combined with gas expansion of a lens capsule. This was surprisingly found to have great advantages, before and during administration of an active agent solution into the capsule. The method according to the invention is defined in claim 1. By employing a novel device according to another aspect of the invention gas is prevented from leaking out from the capsule. The device according to the invention is defined in claim 1.

If gas leakage is observed, indicating that the device is not safely attached at the capsulorhexis, it will be possible to avoid administration of a toxic agent solution. For some devices the gas expansion also strengthen the device-capsule contact by exerting a gas pressure to the device which in turn is pressed against the capsular surface because of the lower pressure in the anterior chamber.

In one embodiment of the device according to the invention the device is a plug adapted to seal against the anterior surface of the lens capsule.

In another embodiment the device for sealing the capsule is a tip of a probe with irrigation and aspiration capabilities.

In a third embodiment the device for sealing the capsule is a tube with a leakage preventing channel, and having the capability of permitting different devices to be introduced into the capsule without leakage of a gas or an agent solution from the capsule. Optionally, the tube cam be composed of several lumens.

In a further aspect of the invention, there is contemplated the use of low doses in the treatment of lens epithelial cells. Compared to prior art methods the dose can be reduced by a factor 5-10 and even 20 in certain circumstances. In a preferred embodiment a detergent is included in the agent solution. Thereby, the administration of the solution to the lens epithelial cells over/at the entire capsular surface is improved, which allows a reduction of the dose by a factor 2 or more.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

The present invention will become more fully understood from the detailed description given hereinbelow and the accompanying drawings which are given by way of illustration only, and thus not limitative of the present invention, and wherein

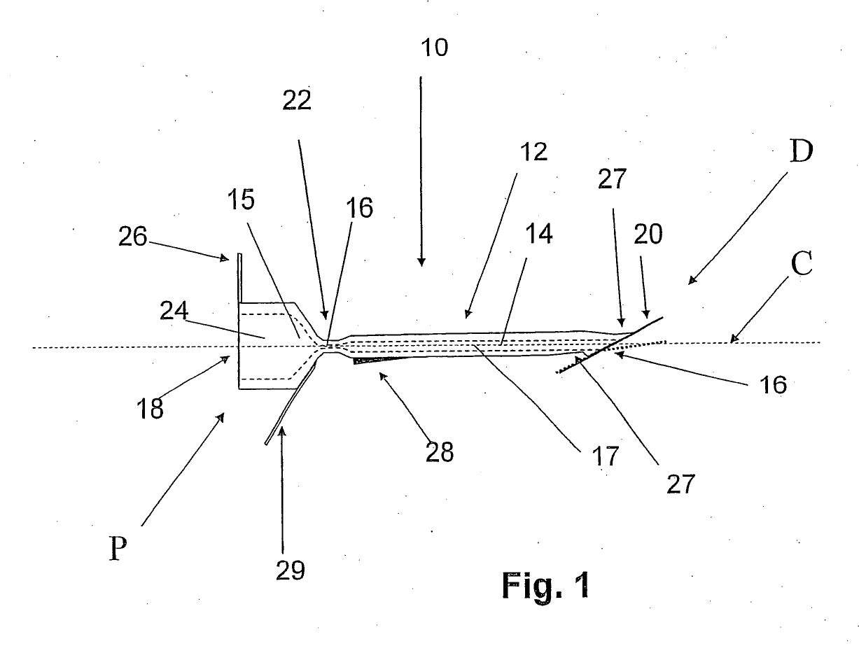

FIG. 1 shows the device according to the invention in a side view;

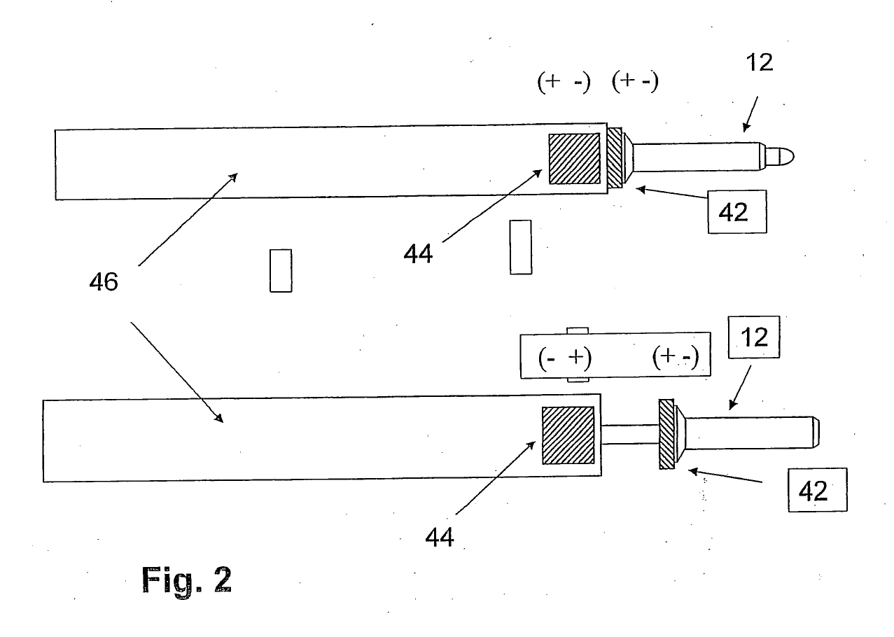

FIG. 2 shows a coupling device for use in the invention; and

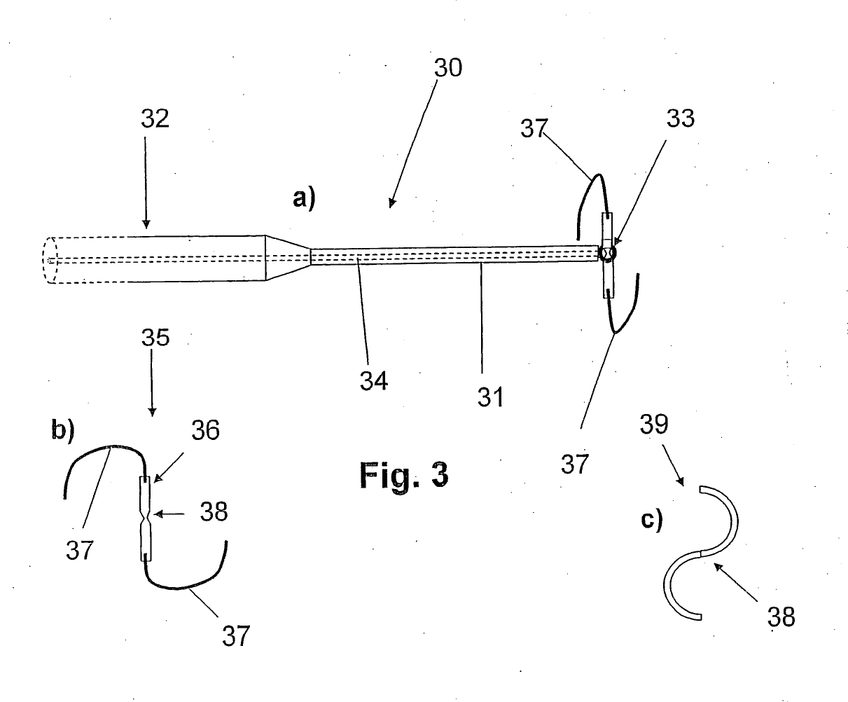

FIG. 3 shows a rotatable device usable with the device according to the invention.

### DETAILED DESCRIPTION OF PREFERRED EMBODIMENTS

The present invention relates to a method suitable for use in eye surgery, and to a specific device and accessories connectable to the device and/or useable together with said device.

The method is based on a general principle, for which patent is applied in PCT/SE02/00854, that is based on the use of e.g. gas to expand a lens capsule and keep it expanded, for the purpose of performing various surgical procedures inside the capsule, e.g. administration of agents for the purpose of killing residual lens epithelial cells, that could cause Posterior Capsular Opacification (PCO).

The novel method is an inventive combination of said general principle and the idea of sealing an eye with a sealing device or means, such as the ones mentioned in the background section.

Thus, the invention relates to a method of eye surgery, which comprises as a first step making an incision in the cornea or the sclera in order to gain access to the interior of an eye. If it is desired to remove the lens, it will be necessary to make a capsulorhexis in the lens capsule. Thereafter, the lens is removed from the lens capsule of the eye, e.g. by phacoemulsification (ultra sound or laser), or by irrigation and aspiration or by extra capsular lens extraction (ECLE), or any other suitable method.

When the lens has been successfully removed, it may be desirable to perform a number of different procedures inside the capsule, such as the above mentioned killing and removal of residual cells, by administering an active agent for killing any residual epithelial cells; working the inner surface of the lens capsule with a wiper device to physically release/detach any residual epithelial cells; or wiping the inner surface of the lens capsule with said device to dry out any unwanted residual liquids, or spread an active agent solution over the inner surface.

In order to be able to perform these procedures in a simple and efficient way, it is convenient according to the invention to expand the capsule with a gas. In order that this be possible, the capsule is first sealed with an appropriate sealing means, to provide gas leakage proof sealing. The sealing can be achieved in different ways, all falling within the scope of the invention. I.a. the devices disclosed in the patent applications mentioned in the background are suitable.

However, there is also provided a novel leakage preventive device, by means of which it is equally possible to use a viscoelastic compound for sealing purposes.

### EXAMPLES

### Example 1

### Plug Sealing

General Experimental Procedure, Group A-D

In groups A to D, rabbits (2.0-2.5 kg mixed sex, New Zealand White) are used to illustrate the invention of PCO prophylaxis. Both eyes of the rabbits are used, with a total of 10 eyes per test group. The eyes of the different groups are randomized between left and right eyes, and between first and second eye in the surgical performance.

Twenty minutes before administering the anaesthetic to a rabbit, the pupils are dilated by topical instillation of mydriatika (cyclopentolat 7.5 mg/ml+phenylephrine 2.5 mg/ml). Anesthetic is given by i.m. injection of 2.0 ml ketamin solution (50 mg/ml) and 1.0 ml xylazin solution (20 mg/ml). Iteration is given occasionally by a second i.m. injection of 0.50 ml ketamin solution. An operating microscope is used to observe the surgery. Before surgery, tetrakain is instilled topically (5 mg/ml), as well as a second instillation of mydriatika. The corneal incision is made by a 3.0 mm angled slit knife. 0.1 ml of 1% heparin solution (5000 IU) is injected into the anterior chamber to prevent synechiae. The anterior chamber is then expanded by injection of a viscoelastic solution (10 mg/ml hyaluronate; Healon). A continuous curvilinear capsulorhexis (D=4 mm) is made using a pair of Corydon forceps, if not else is specified. Hydro dissection is made by injection of balanced salt solution (BSS) between the capsule and the crystalline lens. The crystalline lens is removed by phacoemulsification followed by capsular polishing using an anterior segment operating system (Oertli Quinto, Oertli Instrumente AG, Berneck, Switzerland; Setting of a vacuum of 150 mmHg, a flow of 25 ml/min, a bottle height of 70 cm, and <70% phacoemulsification power at the ultrasound frequency of 28 kHz.). Balanced salt solution with 1.0 ml heparin solution (5000 IU) added, is used as irrigation solution during the lens tissue removal.

The anterior chamber is refilled with a viscoelastic solution based on 10 mg/ml hyaluronate (Healon), except of specified groups where a viscoelastic solution of 23 mg/ml hyaluronate (Healon5) is used instead.

The agent solution is prepared the minutes before use, formed by a water solution of saponin from Quillaja bark (1 mg/ml) and trypan blue (1 mg/ml). Trypan blue is used to make it possible to identify the agent solution through the operating microscope. Saponin has two important characteristics, that has detergent and cell lysis capabilities.

After the performed treatment according to Groups A to D presented below, the rabbits are killed by i.v. injection of 5 ml pentobarbital solution (100 mg/ml). The eyes are enucleated, fixated in 2.5% glutaraldehyde, and embedded in historesin for histological evaluation.

Group A: Plug Sealing and Gas Expansion

A plug according to WO 02/43632 A1 (Maloof) connected to a tube with several lumens is attached at the capsulorhexis to seal the capsule, and used in Groups A-D. The capsule is expanded by injection of 0.2 ml gas (air). The gas is introduced through the plug into the capsule via the tube. If no gas leaks out of the capsule, the capsular sealing by the plug is more reliable. Gas leakage indicates that the plug is not properly mounted or that the plug is defect. Prevented gas leakage guarantees a sealed capsule before introducing an active agent solution.

0.03 ml of the active agent solution is applied to the inner surface of the capsule through the tube and the plug. The solution is administrated efficiently along the inner capsular surface by the phenomenon of surface tension. After one minute of exposure, the agent solution is removed by continuous irrigation and aspiration with BSS inside the capsule using an Oertli Quinto operating machine connected to the tube and the plug.

In the histological evaluation, it is observed that the lens epithelial cells are severely damaged by ruptured cell membranes by the effect of saponin, including the cells attached to the anterior surface of the capsule. No damage is observed on any other tissue outside the capsule, such as corneal endothelium, iris, ciliary process or retina.

The small dose and volume of the agent solution is most likely to be enough to cover the entire capsular surface. The observed gas leakage indicates that the administration of the agent solution should be stopped and thereby leakage of the agent solution is prevented.

Group B: Plug Seating without Gas Expansion

A plug connected to a tube with several lumens is attached at the capsulorhexis to seal the capsule. 0.03 ml of the active agent solution is injected into the capsule through the tube and the plug. A small volume of the agent solution accidentally leaks out to the anterior chamber between the plug and the capsule and the toxic agent solution reaches the iris in the anterior chamber. In the rest of the eyes, the administration of the agent solution is successful only in the central region of the capsule. It does not reach peripheral regions of the capsule. Neither is any spreading by surface tension along the inner capsular surface observed. After one minute of exposure, the agent solution is removed by continuous irrigation and aspiration of BSS inside the capsule using the Oertli Quinto operating machine connected to the tube and the plug.

In the histological evaluation, it is observed that the lens epithelial cells are damaged by ruptured cell membranes, but not for cells situated at peripheral regions of the capsule. No damage is observed on other tissues outside the capsule, such as corneal endothelium, iris, ciliary process or the retina, except for tissues, including the corneal endothelium, in the eyes in which leakage has been observed.

The used dose and volume of the agent solution are not enough for efficient distribution to the lens epithelial cells inside the capsule by this technique. There seems also to be a risk of accidental leakage of an agent solution from the capsule, with no previous signals of a leaking capsule, compared to the use of gas expansion of the capsule before the delivery of the agent solution as in Group A.

Group C: Plug Sealing and Expansion by Agent Solution

A plug connected to a tube with several lumens is attached at the capsulorhexis to seal the capsule. 0.2 ml of the active agent solution is injected into the capsule through the tube and the plug, thereby expanding and completely filling the capsule by the agent solution. No spreading of the agent solution by surface tension along the capsular surface is observed. A leakage of agent solution between the plug and the capsule is observed, as a consequence of a failed mounting of the plug to the capsule. The agent solution reaches several delicate tissues in the anterior chamber, including parts of the corneal endothelium.

The agent solution is removed from the capsule after one minute of exposure by continuous irrigation and aspiration of BSS inside the capsule using the Oertli Quinto operating machine connected to the tubes and the plug.

In the histological evaluation, it is observed that the lens epithelial cells are damaged by ruptured cell membranes, also at the anterior capsular surface and at peripheral regions of the capsule. No damage is observed on other tissues outside the capsule, such as corneal endothelium, iris, ciliary process or the retina, except for the eyes in which leakage has been observed, in which tissues, including the corneal endothelium, has been injured. The dose and volume of the agent solution used is enough for efficient distribution to the lens epithelial cells inside the capsule by this technique. However, it seems to be a risk of accidental leakage of the agent solution from the capsule, and with no previous signals of the leaking capsule, which is given by gas expansion demonstrated in group A.

Group D: Plug and Viscoelastic Seating and Gas Expansion

Instead of a viscoelastic solution of low concentration hyaluronate (10 mg/ml; Healon), as being used in Group A, a viscoelastic solution of high concentration hyaluronate (23 mg/ml; Healon5) is introduced into the anterior chamber after lens removal.

A plug connected to a tube with several lumens is attached at the capsulorhexis to seal the capsule. The capsule is expanded by injection of 0.2 nil gas (air). The gas is introduced through the plug into the capsule via the tube. Gas is prevented to leak out of the capsule, as the sealed capsule is highly reliable, by using both a plug and a viscoelastic solution, indicating that an active agent solution can be introduced without any harm.

0.03 ml of the active agent solution is applied to the inner surface of the capsule through the tube and the plug. The solution is administrated efficiently along the inner capsular surface by the phenomenon of surface tension, enhanced by the detergent saponin. After one minute of exposure, the agent solution is removed by continuous irrigation and aspiration with BSS inside the capsule using an Oertli Quinto operating machine connected to the tube and the plug.

In the histological evaluation, it is observed that the lens epithelial cells are severely damaged by ruptured cell membranes by the effect of saponin, including the cells attached to the anterior surface of the capsule. No damage is observed on any other tissue outside the capsule, such as corneal endothelium, iris, ciliary process or retina.

Results of Group A to D

| | | | | | | Agent | Observed | | |
|---|---|---|---|---|---|---|---|---|---|
| | Plug | Gas | Agent | | Gas | solution | spread of | Observed | Treatment |
| Group | sealing | expansion | solution | | leakage | leakage | solution | injuries | of LEC |
| A | Yes | Yes | 0.03 | ml | Sometimes | None | entire | None | Good |
| | | | | | | | capsular | | |
| | | | | | | | surface | | |
| B | Yes | No | 0.03 | ml | — | Sometimes | central | Sometimes | Poor |
| | | | | | | | capsular | but small | |
| | | | | | | | region | | |
| C | Yes | No | 0.2 | ml | — | Sometimes | filled | Sometimes | Good |
| | | | | | | | capsule | and severe | |
| D* | Yes | Yes | 0.03 | ml | None | None | entire | None | Good |
| | | | | | | | capsular | | |
| | | | | | | | surface | | |

D* = The anterior chambers are filled with viscoelastic solution of 23 mg/ml hyaluronate (Healon5, Pharmacia, Uppsala, Sweden).

The conclusion is, in respect of safety and treatment efficacy, to preferably use the method of group D. That is to use plug sealing and a viscoelastic solution with high concentration of hyaluronate (23 mg/ml) within the anterior chamber, combined with a gas expanded capsule and a low dose and volume of the agent solution.

### Example 2

### Tip Sealing

This experiment is similarly performed according to the description presented in Example 1. This means that groups E to H in this experiment correspond to the groups A to D in Example 1, except for making a smaller capsulorhexis (D=1.5 mm), using a lens removal system for small incision surgery, a device with an impeller (Ref. Baush & Lombs instrument, with the Catarex System) instead of using the Oertli Quinto operating machine, and using a tip instead of a plug to seal the capsule. The instrument was equipped with additional lumens for gas and agent solution delivery.

Results of group E to H

| | | | | | | Agent | Observed | | |
|---|---|---|---|---|---|---|---|---|---|
| | Tip | Gas | Agent | | Gas | solution | spread of | Observed | Treatment |
| Group | sealing | expansion | solution | | Leakage | leakage | solution | injuries | of LEC |
| E | Yes | Yes | 0.03 | ml | Sometimes | None | entire | None | Good |
| | | | | | | | capsular | | |
| | | | | | | | surface | | |
| F | Yes | No | 0.03 | ml | — | Sometimes | central | Sometimes | Poor |
| | | | | | | | capsular | but small | |
| | | | | | | | region | | |
| G | Yes | No | 0.2 | ml | — | Sometimes | filled | Sometimes | Good |
| | | | | | | | capsule | and severe | |
| H* | Yes | Yes | 0.03 | ml | None | None | entire | None | Good |
| | | | | | | | capsular | | |
| | | | | | | | surface | | |

H* = The anterior chambers are filled with viscoelastic solution of 23 mg/ml hyaluronate (Healon5, Pharmacia, Uppsala, Sweden).

The conclusion is, in respect of safety and treatment efficacy, to preferably use the method used in group H. That is to use tip sealing and a viscoelastic solution with high concentration of hyaluronate (23 mg/ml) within the anterior chamber, combined with a gas expanded capsule and a low dose and volume of the agent solution.

### Example 3

### Tube Sealing

This experiment is similarly performed according to the description presented in Example 1. This means that groups I to L in this experiment correspond to the groups A to D in Example 1, except for making a smaller capsulorhexis (D=1.5 mm), using a lens removal system for small incision surgery, a device with an impeller (Ref. Baush & Lombs instrument) instead of using the Oertli Quinto operating machine, and using a tube with a leakage preventive canal, instead of a plug to seal the capsule. In this example no additional lumens were included in the impeller device.

Results of group I to L

| | | | | | | Agent | Observed | | |
|---|---|---|---|---|---|---|---|---|---|
| | Tube | Gas | Agent | | Gas | solution | spread of | Observed | Treatment |
| Group | sealing | expansion | solution | | Leakage | leakage | solution | injuries | of LEC |
| I | Yes | Yes | 0.03 | ml | Sometimes | None | entire | None | Good |
| | | | | | | | capsular | | |
| | | | | | | | surface | | |
| J | Yes | No | 0.03 | ml | — | Sometimes | central | Sometimes | Poor |
| | | | | | | | capsular | but small | |
| | | | | | | | region | | |
| K | Yes | No | 0.2 | ml | — | Sometimes | filled | Sometimes | Good |
| | | | | | | | capsule | and severe | |
| L* | Yes | Yes | 0.03 | ml | None | None | entire | None | Good |
| | | | | | | | capsular | | |
| | | | | | | | surface | | |

L* = The anterior chambers are filled with viscoelastic solution of 23 mg/ml hyaluronate (Healon5, Pharmacia, Uppsala, Sweden).

The conclusion is, in respect of safety and treatment efficacy, to preferably use the method used in group L. That is to use tube sealing and a viscoelastic solution with high concentration of hyaluronate (23 mg/ml) within the anterior chamber, combined with a gas expanded capsule and a low dose and volume of the agent solution.

### Example 4

### Viscoelastic Solution Sealing

This experiment is performed in accordance with the description of the General Experimental Procedure presented in Example 1, but with new methods for the groups M to Q specified below.

Group M: Viscoelastic Solution Sealing without Gas Expansion

0.03 ml of the active agent solution is injected into the capsule. The solution does not spread to peripheral regions of the capsule. Neither is any spreading of the solution by surface tension along the inner capsular surface observed. Minor leakage of agent solution into the anterior chamber is also observed, whereas the solution reaches delicate tissues such as the iris. After one minute of exposure the agent solution is removed by continuous irrigation and aspiration of BSS inside the capsule using the Oertli Quinto operating machine and an I/A tip.

In the histological evaluation, it is observed that lens epithelial cells close to the capsulorhexis are damaged by ruptured cell membranes, but the treatment is incomplete. Damages are observed on other tissues of the anterior chamber, such as the iris.

The distribution of the agent solution is not efficient by this technique to treat all lens epithelial cells inside the capsule. Leakage of an agent solution from the capsule to the anterior chamber is evident by this technique.

Group N: Viscoelastic Solution Sealing without Gas Expansion

0.2 ml of the active agent solution is injected into the capsule. However, the solution does not spread to peripheral regions of the capsule. Neither is any spreading of the solution by surface tension along the inner capsular surface observed. Severe leakage of agent solution into the anterior chamber is also observed, whereas the solution reaches delicate tissues such as the corneal endothelium. After one minute of exposure the agent solution is removed by continuous irrigation and aspiration of BSS inside the capsule using the Oertli Quinto operating machine and an I/A tip.

In the histological evaluation, it is observed that lens epithelial cells close to the capsulorhexis are damaged by ruptured cell membranes, but the treatment is incomplete. Damages are observed on other tissues of the anterior chamber, such as corneal endothelium and the iris.

The distribution of the agent solution is not efficient by this technique to treat all lens epithelial cells inside the capsule. Leakage of an agent solution from the capsule to the anterior chamber is evident by this technique.

Group 0: Deficient Viscoelastic Solution Sealing and Gas Expansion

0.2 ml gas (air) is injected into the capsule. It is observed that the gas leaks out of the capsule into the anterior chamber, indicating a n-on-successful sealing of the capsule. The conclusion is that a viscoelastic solution of 10 mg/ml hyaluronate (Healon) is not enough to prevent gas leakage. 0.03 ml of the active agent solution was injected into the capsule. The leakage of agent solution into the anterior chamber is observed, and is enhanced by remaining gas bubbles inside the anterior chamber. The agent solution is removed after one minute of exposure by continuous irrigation and aspiration of BSS inside the capsule using the Oertli Quinto operating machine and an I/A tip.

In the histological evaluation, it is observed that the lens epithelial cells close to the capsulorhexis are damaged by ruptured cell membranes, but the treatment is incomplete in peripheral regions of the capsule. Some damages are observed on other tissues of the anterior chamber, such as corneal endothelium and the iris.

The distribution of the agent solution is not efficient to treat all lens epithelial cells inside the capsule, and leakage of the agent solution from the capsule to the anterior chamber is evident using this technique.

Group P: Viscoelastic Solution Sealing (23 mg/ml) and Gas Expansion

0.2 ml gas (air) is injected into the capsule. It is observed that the gas does not leak out of the capsule into the anterior chamber, indicating a successful sealing of the capsule by the viscoelastic solution of 23 mg/ml hyaluronate. 0.03 ml of the active agent solution is injected into the capsule. The observation through the operating microscope is similar to Group A, Example 1. The agent solution is removed after one minute of exposure by continuous irrigation and aspiration of BSS inside the capsule using the Oertli Quinto and an I/A tip. In the histological evaluation, it is observed that the lens epithelial cells are severely damaged by ruptured cell membranes, including the cells attached to the anterior surface of the capsule, similar to the results of Group A, Example 1. No damages is observed on any other tissue outside the capsule, such as corneal endothelium, iris, ciliary process or the retina.

The small dose and volume of the agent solution is most likely to be enough to cover the entire capsular surface.

Group Q: Viscoelastic Solution Sealing (25 mg/ml) and Gas Expansion

This experimental group is performed as group L, but a viscoelastic solution of higher concentration of hyaluronate (25 mg/ml; Microvisc Phaco) is used instead to fill the anterior chamber. The observation indicates a slight improvement of preventing leakage of agent solution when using a viscoelastic solution of 25 mg/ml hyaluronate than of 23 mg/ml.

Results of group M to Q

| | Viscoel. | | | | | Agent | Observed | | |
|---|---|---|---|---|---|---|---|---|---|
| | solution | Gas | Agent | | Gas | solution | spread of | Observed | Treatment |
| Group | sealing | expansion | solution | | leakage | leakage | solution | injuries | of LEC |
| M | Yes | No | 0.03 | ml | — | None | central | Yes, but | Poor |
| | 23 mg/ml | | | | | | capsular | small | |
| | | | | | | | region | | |
| N | Yes | No | 0.2 | ml | — | None | central | Yes | Medium |
| | 23 mg/ml | | | | | | capsular | | |
| | | | | | | | region | | |
| O* | Yes | Yes, but | 0.03 | ml | Yes, | Yes | central | Yes, but | Poor |
| | 10 mg/ml | failed | | | severe | | capsular | small | |
| | | | | | | | region | | |
| P | Yes | Yes | 0.03 | ml | No | Sometimes | entire | Sometimes | Good |
| | 23 mg/ml | | | | | | capsular | but small | |
| | | | | | | | surface | | |
| Q** | Yes | Yes | 0.03 | ml | No | Few | entire | Few_and | Good |
| | 25 mg/ml | | | | | | capsular | small | |
| | | | | | | | surface | | |

O* = The anterior chambers are filled with viscoelastic solution of 10 mg/ml hyaluronate (Healon).
Q** = The anterior chambers are filled with viscoelastic solution of 25 mg/ml hyaluronate (Microvisc Phaco).

The conclusion is, in respect of safety and treatment efficacy, to preferably use the method used in group Q. That is to use tip sealing and a viscoelastic solution with high concentration of hyaluronate (25 mg/ml) within the anterior chamber, combined with a gas expanded capsule and a low dose and volume of the agent solution.

### Example 5

### Worse Cases of Agent Solution Administration

This experiment is performed in accordance with the description of the General Experimental Procedure presented in Example 1, but with the groups R and S specified below.

Group R: Low Dose Failure

0.03 ml of the agent solution is placed at the capsulorhexis to simulate worse case of a low dose agent solution injection. It is observed that the agent solution is spread locally around the spot of injection, reaching to the iris. Directly, irrigation and aspiration is performed by a I/A tip inside the anterior chamber. After 4 seconds, at a flow of 25 ml/min, the 0.03 ml agent solution is quickly diluted with a factor of 50 or higher, and removed by aspiration at an equivalent proportion. The outcome is evaluated by histological examination, in which it is observed some injuries to the iris and to the corneal endothelium close to the corneal incision.

Group S: High Dose Failure

0.2 ml of the agent solution is placed at the capsulorhexis to simulate worse case of a high dose agent solution injection. It is observed that the agent solution is spread in a far more extent than in the eyes in Group N, reaching the corneal endothelium and the iris. After 4 seconds, at a flow of 25 ml/min, the 0.2 ml agent solution is diluted with a factor of 8 or higher, and removed by aspiration at an equivalent proportion. The outcome is evaluated by histological examination, in which it is observed severe injuries to the iris and to the corneal endothelium, most extensively at the corneal incision.

Results of Group R and S

| | Viscoel. | | | | Observed | | | Histologic |
|---|---|---|---|---|---|---|---|---|
| | solution in | Gas | Agent | | spread of | | Dilution | indication |
| Group | AC | expansion | solution | | solution | I/A in AC | factor* | of injuries |
| R | Yes | No | 0.03 | ml | local | 4 sec | 50 | Yes, some |
| | | | | | region in | | | |
| | | | | | AC | | | |
| S | Yes | No | 0.2 | ml | more | 4 sec | 8 | Yes, |
| | | | | | extensively | | | severe |
| | | | | | in AC | | | |

*= The effect of aspiration has not been considered, which would level up the dilution factor even further.

For safety reason, the dose of GroupR, low dose and volume, should preferably be used.

Now the devices forming part of the inventive concept and usable with the method according to the invention will be described in detail with reference to the drawings.

Now the leakage preventive device according to the invention will be described in closer detail with reference to FIGS. 1-3.

A leakage preventive device has been found to facilitate and increase the safety to perform manipulations within a lens capsule and to administer gases and solutions into the lens capsule, especially when the surgery includes a lens capsule expanded by a gas.

The leakage preventive device allows the introduction of gases, solutions and different devices (surgical instruments, tools, wipers, patches, absorbing pieces, intraocular implants, intraocular lens molding materials and so forth), and prevent leakage of gases and solutions out of the lens capsule, preferably simultaneously. This is especially important when the lens capsule is expanded by a gas.

Overall Description

The leakage preventive device 10, shown in FIG. 1, having a proximal end P and a distal end D, includes a tube 12 with a proximal portion and a distal portion and a central axis C. Furthermore, for purpose of ease of understanding, the device will be described as having an upper half and a lower half, whereby the dividing plane is through the device in its longitudinal extension, and upper and lower is with respect to the orientation of the device as shown in FIG. 1. The tube is hollow with one or several lumens 14 extending between the proximal end and the distal end. At least one lumen is a leakage preventive lumen. The leakage prevention is achieved by one or several barriers 16 within the lumen 14, the details of which will be elucidated in detail below. The proximal end includes a funnel 18 which enables and facilitates implantation of the leakage preventive device, and also the connecting of different secondary devices and/or the introduction of other devices into the leakage preventive lumen.

In a preferred embodiment the distal end includes a lid membrane 20 which is adapted to close the capsulorhexis opening of the lens capsule. The lid membrane 20 is preferably separable from the tube 12 of the leakage preventive device, thereby providing a closure for the capsulorhexis opening even after the leakage preventive device has been removed from the eye. This is important when an intraocular lens implant is molded within the lens capsule by an injected lens forming fluid. By providing a circumferential groove or depression in the tube 12 near said lid membrane, it will be an easy matter to cut off the membrane when the operation is finished. The lid 20 can have a cut out or slit enabling passing of devices and gas through said lid.

The leakage preventive device is preferably made of one or several different materials such as polyurethane, silicone, hydroxymethylmethacrylate, polymide, polymethylmethacrylate, polyethylene, polyester, polystyrene, polypropylene, polytetrafluorethylene, ethylene-vinyl-acetate or equivalent. Furthermore the material may have adhesive qualities, and preferably it should be pliable, resilient and non-absorbent, i.e. it should not absorb any of the liquids or agents that it may come into contact with during use.

In use, the device is inserted through an incision of the eye, such as a corneal or a scleral incision. When properly located, the proximal end and the funnel are located outside the incision, and the distal portion of the tube is located within the eye. Thereby, the proximal portion of the tube extends through the incision into the anterior chamber, and the distal portion extends within the anterior chamber towards the lens capsule and the distal end is directed to or into a lens capsular opening.

Tube

The leakage preventive device includes a tube 12 having a proximal portion and a distal portion extended between the proximal end and the distal end of the leakage preventive device. The tube may have a cross-sectional dimension of about 0.5 to 3 mm such as about 1.5 mm. The total length of the tube is preferably in the range of 5 mm to 15 mm, such as about 7 mm.

The tube is preferably made of a non-absorbent material such as polyurethane, silicone or equivalent. Furthermore the material may have adhesive properties, and should preferably be pliable and resilient. However, the tube should be stiff enough to avoid undesired wrinkling when being used.

The tube is hollow and includes one or several lumens 14 extending from the proximal end to the distal end. The purpose of the lumens is to allow devices (surgical instruments, intraocular implants and so forth) to be moved through the tube, as well as solutions and gases.

At least one lumen should prevent the leakage of liquids and/or gases outwards through the tube. The purpose is to allow moving different instruments (surgical instruments, cannulas, threads, wipes, drying sticks, tips and so forth) through the tube and preferably at the same time to prevent gases or solutions to leak out, preferably in a simultaneous fashion.

Lumen Barriers

The leakage preventive capability of the lumen is obtained by one or several barriers 16. The barriers can be of different kinds, such as viscoelastic solutions 17 occupying the lumen, in which case the high viscosity makes it possible to provide the barrier. Other possibilities are membranes 20 covering the opening of a lumen, flattened passages 22 within a lumen, narrow passages in a lumen, valves, flap valves just to mention a few possiblities. Barriers such as membranes, flattened passages, narrow passages, valves, flap valves can be positioned at one or both ends of the lumen or somewhere between the proximal end and the distal end. When the barriers are provided as e.g. viscoelastic solutions, flattened passages, narrow passages they can extend along the whole lumen or partial regions of the lumen. It would pertain to the field of the man skilled in the art to provide other kinds of barriers with equivalent function to be used for the same purpose.

Several barriers can be included simultaneously to improve the leakage preventive capability. For example, a viscoelastic solution along the lumen enclosed by a flattened passage at the proximal portion and a flap valve at the distal end, would very efficiently prevent leakage of gases and solutions. Furthermore, the distal and proximal barriers also serve to retain the viscoelastic solution in the lumen when different instruments are moved through the viscoelastic solution.

One important criterion of all barriers is that it should be possible to force secondary devices through the barriers, and that the barrier still would prevent leakage of solutions and gases, preferably simultaneously.

To improve the passing of devices through the barriers, it is preferred that the friction between the inner surface of the lumen and passing devices is low, or at least as low as possible. A viscoelastic solution inside the leakage preventive lumen has the additional advantage of serving as a lubricant.

Funnel

At the proximal end there is a funnel 16 which includes a wide opening, narrowing gradually into the leakage preventive lumen of the tube 12. The funnel 16 has a shape that facilitates the insertion of different instruments into the lumen. Suitably, the shape is a symmetric conical shape 15.

The outer diameter of the wide opening is preferably in the range of 3 to 15 mm, such as 7 mm. It is then gradually decreased to the dimension of the proximal part of the tube. The length of the funnel is preferably in the range of 0.5 to 15 mm, such as 4 mm.

The funnel may have a shape that is suitable for connecting different instruments to the leakage preventive device, such as an irrigation/aspiration device. The funnel may have different connecting options, such as Luer Lock® 24, threads, slots or equivalent.

The funnel 18 can form an angle with respect to the tube in the range 0 to 45°. Preferably the angle is selected such that when the device is properly positioned in the eye, the funnel is perpendicular to the surface of the eye globe at the point of insertion. For ease of handling, e.g. guiding, rotation and holding the device, the angle is 0°. The device will also be easier to manufacture in the latter case. The skilled man will at any rate be able to design the device with a funnel angle that is suitable for a desired purpose.

In one preferred embodiment, the funnel is provided with a gripping means 26, suitably in the form of a short pin or protrusion, located at the periphery of the funnel 18, near or at the opening, and having a length in the range of 2 to 30 mm, preferably 8 mm. The purpose of the gripping means 26 is to allow guiding, holding and/or rotating the leakage preventive device, such as by a pair of forceps. The gripping means can also have a shape that allows fixation of the tube to the eye, such as a connector member (not shown) that can be connected to an eye lid holder or equivalent.

In another preferred embodiment of the device, there are fixation means 29 for temporary fixation of the leakage preventive device to the eye globe, such as loops attached to the funnel, thin flaps extending laterally from the funnel, optionally having holes for sutures, to which a suture easily can be attached for fixation by some stitches to the scleral region of the eye globe. Adhesive elements is also envisaged for attaching to the scleral region.

In another preferred embodiment, the funnel comprises a coupling means, to enable quick and controlled attachment and release of secondary devices to/from the funnel, or other devices introduced into the leakage preventive lumen. The coupling means can comprise magnets, spring driven catchers or equivalent.

In one preferred embodiment, shown in FIG. 2, the coupling means comprises one or several integrated permanent magnets 42 positioned at the wide opening of the funnel, such as a magnetic ring at the edge around the opening. By using the interaction between the different polarities of the magnets (+/−), a secondary device 46 with one or several (electro)magnets 44 can be forced to join the leakage preventive device, by magnetic attraction, or separated from it by a controlled operation, such as by changing the polarity of the electromagnet 44 of the secondary device 46, causing magnetic repelling. For example, the join and release function is valuable for application in implanters designed to, temporary implant a leakage preventive device into an eye.

In still another preferred embodiment, the funnel has a drip edge at the wide opening that prevents any drops of agent solutions to flow from the funnel to the surrounding tissues. For example, a colored agent solution may be easily identified and removed from the funnel.

Catcher

In one preferred embodiment a fixation member 28 (FIG. 1) is provided at the proximal portion of the tube 12. The purpose of the fixation member is to keep the device in a correct position at the incision of the eye during surgery.

The fixation member may be designed as a wedge shaped rim element that is positioned at the lower half of the proximal portion of the tube 12, in a position that will be within the eye, grabbing with its hook end at the posterior surface of the cornea or equivalent region of the eye globe. The wedge is preferably made of the same material as the tube, not excluding the possibility that other materials are possible.

The catcher can be provided by a flattened or narrowed passage 22 of the tube with the capability of serving as a lumen barrier.

It is highly desirable to increase the fixation during any movement (e.g. rotation) of devices within the leakage preventive device. In order to enhance the fixation, the proximal portion of the tube in one preferred embodiment is provided with a fixation portion that includes frosted and/or textured surfaces of the tube, to increase the friction between the device and surrounding tissue. Preferably, portions of the surface or the entire surface of the flattened or narrowed portions of the tube are frosted and/or textured. The frosted surface portions can be continuous or non-continuous with other portions. Various designs of the frosted surface portions exist, for example, they can include stripes, checkerboard, dots, circles, triangles, squares, pentagons, octagons, lattice or virtually any other type of design. The frosting can be provided on the front, back and/or edge surfaces. The frosted and/or textured surface portions can be applied to the proximal tube portion by various methods including molding or surface texturing. Specifically, surfaces of the mold can be frosted or textured (e.g. sandblasting, electrode discharge machining (EDM), shot peening, laser etching chemical etching, sputtering, vapor depositing, etc.). Alternatively or in addition, the surfaces of the proximal tube portion can be frosted or textured by; for example, sandblasting, shot peening, chemically etching, chemically etching portions to become frosted or non-frosted, surface modifying or other various techniques to make portions of the haptic lightly frosted or textured, medium frosted or textured, highly frosted or textured, or frosted or textured to any degree there between. The frosted and/or textured surface portions of the proximal tube portion increase friction in the interface between the proximal tube portion of the leakage preventive device and the corneal incision, to improve the fixation of the leakage preventive device at the corneal incision or equivalent incision of the eye globe.

Lid Membrane

The lid membrane 20, is provided at the distal end of the tube 12, covering the opening thereof, and has an extension such that it covers and overlaps the capsulorhexis. It is preferably provided with means for providing a self closing opening, such as a slit, flap valves, narrowing or so forth. In order to be easily separated from the tube of the leakage preventive device, when the medical procedure is terminated, there is preferably provided a circumferential groove 27 or recess around the tube 12 near the lid 20. The material for the lid is preferably having a light refraction index similar to the material of injectable intraocular lenses, in order that it will not cause any disturbance. Preferably it has an oval shape which makes it easily insertable through a lumen in the tube 12 of the device 10.

Rotatable Device

In order to render the method according to the invention as efficient as possible, e.g. to ascertain that lens epithelial cells be killed at the highest possible rate, or to wipe the inner surface dry, or to administer an agent solution at the interior surface of the lens capsule, physically remove any residual cells that might adhere inside the lens capsule, it would be attractive to be able to access the interior via the leakage preventive device. This can be achieved by providing the leakage preventive device with a rotatable device 30, illustrated in FIG. 3a).

This rotatable device 30 is insertable into the lens capsule via one of the lumens in the tube. Thus, the purpose is to use the device for surgical manipulations within the lens capsule, preferably expanded by a gas, and preferably to prevent leakage of gases and solutions from the lens capsule during such operations. Examples of surgical manipulations to be executed by the device inside the lens capsule are wiping, drying or administrating an agent solution at the interior surface of the lens capsule. The rotatable device is movable within the lumen (such as rotation in both directions and movements along the lumen, into and out of it and through it) and able to be separated from the tube.

Overall Description

The rotatable device 30 includes A) a shaft 31 having a proximal end and a distal end, B) a grip 32 in the proximal end to facilitate movement and rotating forces to the rotatable device and/or solutions and gases, and C) a connector member 33 in the distal end for attachment of accessories. The total length of the rotatable device 30 is longer than the length of the lumen 12, such as 8 mm and above.

In one preferred embodiment the rotatable device includes an extension at the distal end, instead of a connector, comprising a desired accessory, such as a wiper.

Shaft

The rotatable device includes a shaft 31 having a proximal end and a distal end and a central axis extending between the ends. The shaft is homogenous and is preferably formed as a thread or a stick, though it may also be hollow. It is made of a material with torsion resistant properties to allow a transferred rotating force of movement along the shaft. Examples of suitable materials are metal, nylon, polystyrene, polymethylmethacrylate (PMMA) or equivalent material with similar properties. The diameter of the shaft is smaller than the inner diameter of the leakage preventive lumen, preferably a dimension of 0.1 to 1.5 mm, such as 0.3 mm.

In one preferred embodiment, the shaft is flexible, referring to the ability of bending the shaft along the central axis, but still having the torsion resistant properties.

In another preferred embodiment the shaft is hollow including one or several capillary lumens 34 extending from the proximal end to the distal end, preferably having a dimension of about 0.1 to 1 mm, such as 0.2 mm. The purpose of the lumens is to transfer solutions and gases into and out of the lens capsule. In such cases, the lumens at the proximal end should preferably be connected to another device for injecting solutions or gases, or for aspirating those. This device can also be a part of the rotatable device.

Grip

At the proximal end of the shaft there is a grip 32 that facilitates rotation and movements of the shaft 31, by hand or by mechanical force. The grip is preferably formed as a handle, a knob for manual operation. Alternatively, it can be designed such that it enables connecting a secondary device to facilitate or create movements and/or rotation. The grip is preferably made of the same material as the shaft, but other materials are also useable to achieve a grip with said properties. If there is one or more lumens in the shaft, the grip is adapted to fit secondary devices.

Connector Member

At the distal end of the shaft there is provided a connector 33. The shaft 31 and the connector 33 can be made in one piece or they can be made in two separate parts joined together by physical or chemical bonding, such as by glue, friction, threads, splines or the equivalent. The connector enables attachment of accessories to the rotatable device, such as a wiper, a piece of absorbing material and so forth. The connector enables the transfer of movements and rotating forces from the rotatable device to the attached accessory.

In one embodiment, the connector simply comprises a ring member attached to the distal tip of the shaft.

The outer measures of the ring should be smaller than the inner dimension of the lumen, into which the rotatable device is aimed to be positioned. Different accessories can easily be attached to the ring by simply passing the accessory through the ring such that the ring encircles a body portion of said accessory. If made by a suitable material, such as soft silicone, the accessory can be provided with locking rims cooperating with the ring to hold the accessory in place during operation, while still being easily removed again.

In the use of the leakage preventing device, the rotatable device and an accessory, the rotatable device with the attached accessory is forced through the lumen with the barriers of the leakage preventing device. In this procedure the accessory is folded backwards at the lumen entrance of the funnel and the resilient material of the accessory allows it to be stretched and extended inside the lumen. Once it has passed the lumen, the accessory will retain the desired shape within the lens capsule to execute a manipulation, such as delivering an agent solution, wiping the interior surface of the lens capsule and so forth.

Wiper

In one preferred embodiment, the accessory is a wiper 35, shown in FIG. 3b), with a central body 36 and one or several loops 37 extending from the central region 36. Preferably the central body 36 has a narrowed waistline 38, in order to retain the central body 36 in its position in the connector.

In another preferred embodiment, the rotatable device and a specific accessory, such as a wiper, is made in one piece or pieces attached to each other more permanently by physical or chemical binding, such as by glue, friction or equivalent.

In another preferred embodiment, the distal end of the shaft of the rotatable device is formed as a wiper, such as a pre-shaped thread that will return into desired shape once introduced into the lens capsule.

In still another preferred embodiment, the wiper is a type of formable resilient device including a central portion and two loop-type haptic portions. The haptic and the central portions are preferably made of resilient material, for example, polyurethane, polypropylene, polyimide, polymethylmethacrylate (PMMA), silicone elastomer, hydrogel polymer, collagen containing polymer material (e.g. Collamer), organic or synthetic gel compounds, polyurethane elastomer or other suitable biocompatible materials. The haptic and the central portions can be made of the same material or different. The haptic portions are anchored in the lens portion, if not molded in one piece. The anchoring process is such that the haptic portions remain securely anchored in the central portion during wiper movement (such as through a lumen), wiper rotation, and throughout the surgical-use of the wiper.

The haptics are preferably C-shaped, however, other suitable haptic designs can be substituted for the ones shown. For example, the central portion can be provided with several connecting portions located at ends of the central portion, and the haptics can be full loops anchored at both ends to a pair of the connecting portions. Alternatively, the haptic can be a ring connected to the central portion, such as by bridging haptic portions

The haptic portions can have the shape and profile shown in FIG. 3. However, other shapes and configurations of the haptic portions are possible. Other suitable haptic portions are shown in U.S. Pat. Nos. 4,573,998, 4,702,244, and 5,776,191 to Dr. Thomas R. Mazzocco, incorporated herein by reference.

One preferred embodiment according to the present invention utilizes haptic portions made from polyimide sheet material in combination with a collagen containing polymer lens material (e.g. “Collamer”). The “Collamer” material is disclosed in detail in U.S. Pat. Nos. 5,654,349, 5,654,363, 5,654,388, and 5,661,218 to Vladir Feingold and Alexi V. Osipov, incorporated herein by reference. The different parts can preferably be made in one piece by the same material in all parts.

Haptic Portions

The haptics include a pair of stems extending radially from the central portion on diametrically opposed sides, the stems being disposed in a substantially horizontal plane.

In one preferred embodiment, the stems are generally round in cross-section.

Frosted Haptic Surface Portions

In one preferred embodiment the wiper is provided with haptic portions that include frosted and/or textured surfaces. Specifically, portions of the surfaces or the entire surfaces of the haptic portions are frosted and/or textured. The frosted surface portions can be continuous or non-continuous with other portions. Various designs of the frosted surface portions, for example, can include stripes, checkerboard, dots, circles, triangles, squares, pentagons, octagons, lattice or virtually any other type of design including potentially art work. The frosting can be provided on the front, back and/or edge surfaces.

The frosted and/or textured surface portions can be applied to the haptic portions by various methods including molding or surface texturing. Specifically, surfaces of the mold can be frosted or textured (e.g. sandblasting, electrode discharge machining (EDM), shot peening, laser etching-chemical etching, sputtering, vapor depositing, etc.). Alternatively or in addition, the surfaces of the haptic portions can be frosted or textured by, for example, sandblasting, shot peening, chemically etching, chemically etching portions to become frosted or non-frosted, surface modifying or other various techniques to make portions of the haptic lightly frosted or textured, medium-frosted or textured, highly frosted or textured, or frosted or textured to any degree there between.

The frosted and/or textured surface portions of the haptic portions increase friction in the interface between the lens capsule and the haptic portion to increase the extent of wiping capacity to affect lens fiber cells and/or the epithelial cells. It is highly desirable to increase the wiping power during any relevant movement (e.g. rotation) between the wiper and the interior lens capsular surface.

Based on mechanics, it is highly desirable that tip portions of the haptic portions are frosted and/or textured to provide the greatest moment arm for frictional forces between the haptic portions and the lens capsule to improve the wiping capacity by the movement and/or rotation of the wiper within the lens capsule. Further, both sides and the edges of the haptics portions are preferably frosted and/or textured to increase the total surface area that is frosted, and thus increase the amount of frictional resistance to movement of the wiper.

In the context of a one-piece plate-type IOL according to the present invention, a portion of one or both haptic portions can be intentionally colored. The coloration can render the haptics opaque, or partially transmissive (i.e. translucent). The coloration can be used independent of any surface frosting, or in combination therewith. For example, the same surface portions can be both frosted and colored, or different portions can be frosted and not colored while other portions are not frosted, but colored.

The coloration can be utilized to reduce the transmittance of light through the haptic portions, for marking the wiper (e.g. trademark logos, wiper orientation markings, bar codes, etc.).

Wiping and Absorbing Element

In one preferred embodiment, the accessory is an absorbing member 39 made of a piece of an absorbing material, such as bonded cellulose acetate, bonded polyolefin, combinations thereof or equivalent material with an absorbing capability. The function is to dry the interior of the lens capsular bag by absorbing and removing liquids, such as irrigation solution or an agent solution. The absorbing member 39 is movable through the lumen, into and out of it and can be separated from the tube and disposed to be exchanged for a new absorbing piece until the lens capsule has been dried or liquids have properly been removed.

It can be provided in the form of an elongated stick or pin, preferably S-shaped, for providing a good sweeping action inside the capsule during operation. The diameter of the absorbing member is smaller than the inner diameter of the lumen of the tube, preferably 0.2 to 1.5 mm, such as 0.5 mm. The diameter of the shaft or the thread to which the stick is attached to, should be smaller than the inner diameter of the lumen, similar as for the stick. The stick and the shaft/thread is preferably attached to each other by physical or chemical bonding, such as by glue, friction etc.

In one preferred embodiment the absorbing element is made of an expandable material, such as foam-rubber or foam plastic, bonded cellulose acetate, bonded polyolefin, that is able to be compressed for passing the leakage preventive tube, and able to be expanded inside the lens capsule for wiping and absorbing solutions inside the capsule.

In one preferred embodiment, the absorbing element is pretreated with an agent solution that can be wiped on the interior surface of the lens capsule.

A great advantage with the method according to the invention is that it is possible to employ a low dose of agent solution and a small administered volume.

The following doses correspond to experimental treatments in rabbits. The principle is applicable in to other species, such as human beings.

In a normal situation, 0.1 to 0.2 ml of the agent solution is needed to fill the capsule completely without the use of a gas expanded capsule. The active agent can be selected from cell killing, or cell passivating compounds, such as 5-fluorouracil, doxorubicin, gene modifying substances, saporin just to mention a few possible alternatives.

Additives that enhances the function of the agent solutions are a dye (e.g. Trypan blue, fluorescin) to render the solution easily visible, detergents (e.g. Tween® (polyoxyethylene (20) sorbitan mono-oleate), SDS, saponin) to improve the spreading of the solution inside the lens capsule over the inner surface thereof.

The volume is dependent on the size of the capsule. This correspond to a dose of 10 to 20 mg saponin when administering a solution with a saponin concentration of 100 mg/ml. Surprisingly, it was found that much lower doses, a dose of 1 to 5 mg saponin, is enough to achieve the same result or even better. This corresponds to 0.01 to 0.05 ml of the agent solution when administering a solution with a saponin concentration of 100 mg/ml. In conclusion, the necessary dose in a gas expanded capsule has surprisingly been found to be 10 to 50% of the necessary dose without the gas. A saponin concentration of 100 mg/ml was found to have good efficacy in rabbit experiments. Most likely the necessary concentration is much lower, but a dose titration curve has not yet been made. It seems obvious to find the lowest possible concentration to be used by a dose titration curve. The gain of using a gas expanded capsule will be similar at even lower concentrations of an active agent, such as saponin.

It is also possible to use other substances in similar proportions as those illustrated above, and obtain the benefit of the technique according to the invention, namely to be able to use lower doses over all. Examples of other substances are doxorubicin, 5-flurouracil, saporin.

Furthermore, it was surprisingly found that including a detergent in the agent solution, improved the administration of the solution to the lens epithelial cells. If the capsular surface is dry when administering the agent solution, approximately 0.05 ml is needed to distribute an agent solution over the entire surface. By combining the solution with a detergent this volume can be reduced to 0.02 ml or less, corresponding to the same reduction in the given dose of the active agent.

The addition of a viscoelastic substance to the agent solution, 0.05%-25%, preferably 0.05-5%, suitably 0.05-3%, makes the risk of leakage less pronounced, and will enable control of the behavior of the agent solutions distribution inside the eye and its lens capsule.

A low concentration of hyaluronic acid, for example, 0.5% hyaluronic acid (based on dilution of Healon (Pharmacia, Sweden), with a MW of 4 000 kDa.) slows down the spreading of the agent solution.

A higher concentration of hyaluronic acid, for example, 2.0% hyaluronic acid (based on dilution of Healon5 (Pharmacia, Sweden), with a MW of 4 000 kDa.) makes the agent solution very rigid or stiff and can therefore only be actively distributed, i.e. spread out at the inner surface of the lens capsule with a specially designed device.

Other viscoelastic substances that could be useful are thermo-elastic substances (e.g. polyoxyethylene-polyoxypropylene block copolymers; an example being Lutrol®); and methyl cellulose.

Proposed Composition with Hyaluronic Acid

0.5% hyaluronic acid

0.1% trypan blue

0.5% saponin

hypotonic solution

The solution is applied directly to the inner surface of the lens capsule, and spread along the surface with a rubbing device. The equilibrium between the spreading function of the surfactant and the controlling function of the viscoelasticum is important for obtaining the most efficient spread and most effective prevention of leakage out of the capsule.

The small doses and volumes have major advantages from a safety aspect, namely to avoid injuries to delicate tissues of the eye, such as the corneal endothelium. For example, a small dose is quickly diluted by irrigation solution to a less harmful concentration.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A device (10) for preventing leakage of gas from a lens capsule of an eye, to which a capsulorhexis has been made, during manipulations performed inside said lens capsule, comprising
a tube (12) having a proximal (P) and a distal (D) end, and at least one lumen (14) extending between said ends, said tube (12) being adapted to be located with its distal end positioned in the vicinity of said capsulorhexis of the lens capsule of the eye, and with its proximal end positioned outside the eye during said manipulations;
at least one barrier member (16; 17) provided with the tube (12) such that gas that has been introduced into the lens capsule is prevented to escape therefrom.

2. The device according to claim 1, wherein there is provided on the distal end of the tube a thin, flexible flat lid member (20) covering the distal opening of the tube, and having an extension such that once in place inside the lens capsule, it covers and overlaps the capsulorhexis, thereby forming a seal.

3. The device according to claim 2, wherein the lid member (20) is provided with a slit.

4. The device according to claim 1, comprising a plurality of lumens.

5. The device according to claim 1, wherein at least one lumen is filled with a visco elastic substance (17), forming a barrier against gas leakage.

6. The device according to claim 1, wherein the device is provided with a plurality of barrier members (16).

7. The device according to claim 1, further comprising a funnel (18) provided at the proximal end of the tube (12).

8. The device according to claim 7, wherein the funnel (18) forms an angle with the tube (12).

9. The device according to claim 1, wherein the tube (12) is provided with a fixation member (22; 28), ascertaining a correct position of the device once placed in its operative position.

10. An active agent solution for the treatment of an eye to prevent Posterior Capsular Opacification (PCO), comprising as the active agent a compound selected from cell killing, or cell passivating compounds; additives that enhance the function of the agent solution, selected from a dye to render the solution easily visible; detergents to improve the spreading of the solution inside the lens capsule over the inner surface thereof; and saponin in a concentration of less than 100 mg/ml, preferably less than 50 mg/ml, more preferably less than 30 mg/ml.

11. An active agent solution according to claim 10, wherein the active agent is selected from saponin, 5-fluorouracil, doxorubicin, gene modifying substances, saporin, SDS, Tween®.

12. An active agent solution according to claim 10, wherein the dye is selected from Trypan blue, fluorescein.

13. An active agent solution according to claim 10, wherein the detergent is selected from Tween®, SDS, saponin.

14. An active agent solution according to claim 10, further comprising a viscoelastic compound.

15. An active agent solution according to claim 15, wherein the viscoelastic compound is selected from hyaluronic acid, thermo-elastic substances, and methylcellulose.

16. An active agent solution according to claim 10, comprising a hypotonic solution.

17. An active agent solution according to claim 10, comprising 0.5% hyaluronic acid, 0.1% trypan blue, 0.5% saponin, and hypotonic solution.

18. A method of eye surgery, comprising the steps of:
making an incision to gain access to the interior of an eye;
making a capsulorhexis in the lens capsule;
removing the lens from the lens capsule of an eye;
sealing the capsulorhexis with a sealing means/device, to vide gas leakage proof sealing;
expanding the lens capsule with a gas;
performing a desired operation inside said expanded lens capsule.

19. The method according to claim 18, wherein the incision is a scleral or corneal incision.

20. The method according to claim 18, wherein the lens is removed by phacoemulsification, by irrigation and aspiration or by extra capsular lens extraction (ECLE).

21. The method according to claim 18, wherein the gas is selected from inert gases, such as air, nitrogen, noble gases.

22. The method according to claim 18, wherein the operation that is performed comprises administering an active agent for killing and/or passivating any residual epithelial cells.

23. The method according to claim 18, wherein the operation that is performed comprises working the inner surface of the lens capsule with a wiping device to physically release/detach any residual epithelial cells.

24. The method according to claim 18, wherein the operation that is performed comprises wiping the inner surface of the lens capsule with a wiper device to dry out any unwanted residual liquids, and/or to spread agent solution over the surface.

25. The method according to claim 24, wherein the wiper device comprises absorbent members.
